(19) United States
(12) Patent Application Publication
(10) Pub. No.: US 2007/0098856 A1
(43) Pub. Date: May 3, 2007

(54) Mealtime eating regulation device

(76) Inventor: Jacques LePine, Paris (FR)

Correspondence Address: LEPINE, 10 rue d'Alger, Paris, 75001, FR

(21) Appl. No.: 11/640,522

(22) Filed: Dec. 18, 2006

(30) Foreign Application Priority Data
May 4, 2005 (WO) PCT/FR05/01118
Apr. 17, 2004 (FR) FR/0405342

(51) Int. Cl. G01N 33/02

(52) U.S. Cl. 426/231

(57) ABSTRACT

The present invention relates to a device of regulation of the food characterized in that it includes/understands a device of measurement of the catch of meal which includes/understands in particular a measurement of the chewing and/or swallowing (20) and/or a measurement of the pin (10) of the gesture of food catch, measurements being then treated by a terminal (30) which restores an analysis with the user of it.

The present invention relates to a meal regulation device which measures the various moves of the user during the meal and which then gives to the user a teaching feedback of these moves. Measured moves being for example that of the hand which brings food into the mouth, that of the chewing with the jaw, and that of the swallowing with the throat.

### BACKGROUND OF THE INVENTION

At the time being, there exists yet no food regulation device which works through the moves displayed when eating during the meal. What exits is either through the quality of food itself with selections and assembly of food, or either by a repair of the damages of food with repairs on the body by surgery or by electro stimulation. The specific period of the meal during which the consumer will absorbs food is however critical. The kinetics of catch of meal, duration, quantity, frequency, the quality of the chewing of food and swallowing has influence on the health of the consumer.

A purpose of the invention is to propose a device of regulation which intervenes by measuring moves of the user during the meal.

An purpose of the invention is to propose a device which gives a feedback to the consumer, realtime during the meal or after the meal, with feedback information on the hand to the mouth, chewing and swallowing moves.

A purpose of the invention is to teach the user ways to better apprehend the kind of his moves, their defects and then gradually to help him progress in his way of feeding. The experience feedback being carried out by means of an expert software which the means either of a warning device, or of a comparison of behavior with other consumers in order to set a program of progression towards a better way of feeding.

A purpose of the invention is to propose a device of regulation which is light, mobile and which can be easily manufactured. The invention finding at the same time an application in a cheap line of goods, but also another application in a range of top-of-the-range price.

A purpose of the invention is to propose a communicating device which easily easily corresponds for the follow-up of progress, with its tutor or with other users presenting the same profile of problems of nutritive kinetics.

A purpose of the invention is to be propose a device assistant to various users' questions such as a fine control of the weight and a good being of life, such as heavier medical problems of overweight or obesity, or such as medical cases of deformation of the digestive system.

### BRIEF DESCRIPTION OF THE INVENTION

A principal aspect of the invention comprises a device which follows up specific moves of the user during the meal.

An aspect of the invention comprises a device which measures the chewing of the consumer. This device for example being placed in an auricle carried by the consumer.

In an aspect, the invention comprises a device which measures the swallowing. In an aspect, the invention comprises a device of which measures of the gesture of the hand to the mouth. This device being for example built-in in the handle of cover, fork, knife, spoon, or in another version in a watch with the wrist. This device includes a device of the known type of dynamic localization, for example with a miniature gyroscope, or an accelerometer which indicates at the same time the position in the space of cover, its acceleration of movement, but also its angular declinivity compared to the vertical.

In an aspect of the invention, the gesture measure devices measures are equipped with a chip of processing data and with a transmitter which transmits the data on a distant terminal provided with a software of processing of these data. The terminal thus making it possible to the user to read and analyze, realtime during the meal or as well at the end of the meal the different kinetic measures: speed of meal, numbers of fork into the mouth, quantity of food, quality of chewing of food. This restitution being able to be carried out by means of a restitution by graphic table or alarm, directly through a specific nearby terminal or through a distant Internet portal with a human assisted or software analysis.

In an aspect the invention also comprises a real time warning system which informs the user during the meal when entering a critical zone of the necessity to speed up or slow down his blow of fork, his chewing or his swallowing.

### BRIEF DESCRIPTION OF THE SEVERAL VIEW OF THE DRAWINGS

The annexed figures represent a particular mode of the invention on which:

FIG. 1 represents a feeding move measuring device according to an aspect of the invention

the FIG. 2A represents a chewing measuring device according to an aspect of the invention

the FIG. 2B represents a chewing measuring device on a consumer according to an aspect of the invention

FIG. 3 represents a processing circuit of data according to the invention

FIG. 4 represents a data restitution according to the invention

the FIGS. 5a, 5b and 5c represent three more particular forms of connection between feeding move measuring devices and data processing device

### DETAILED DESCRIPTION OF THE INVENTION

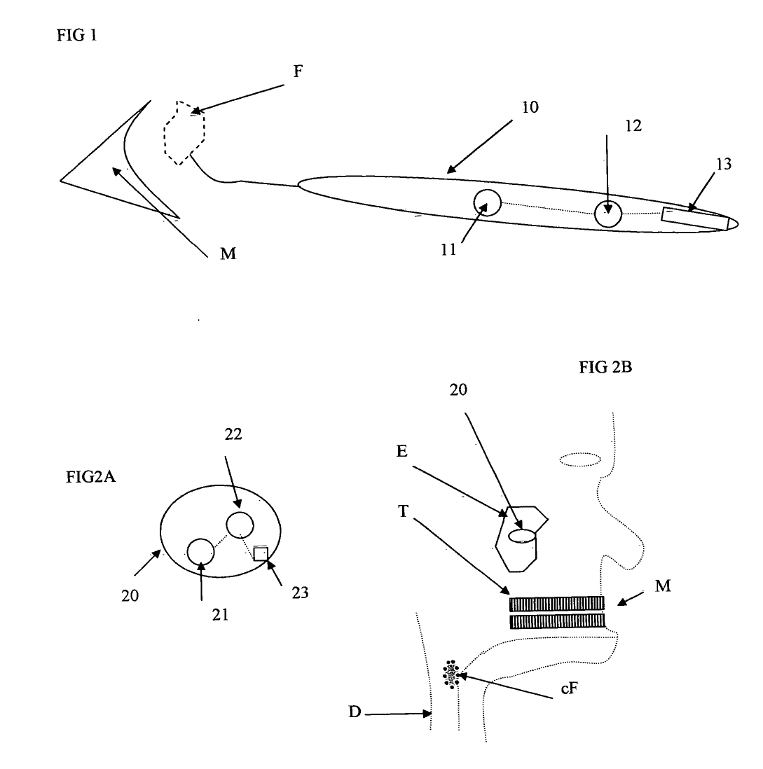

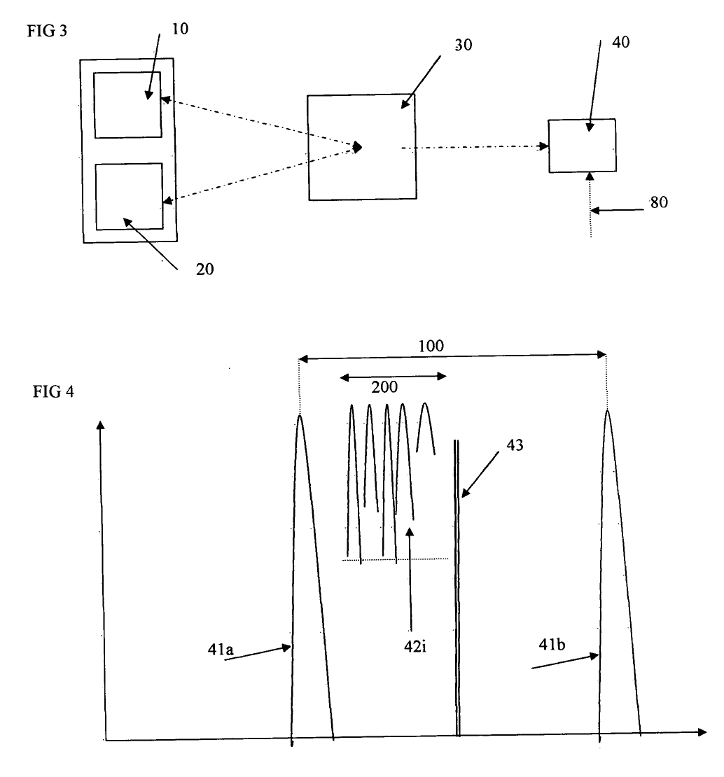

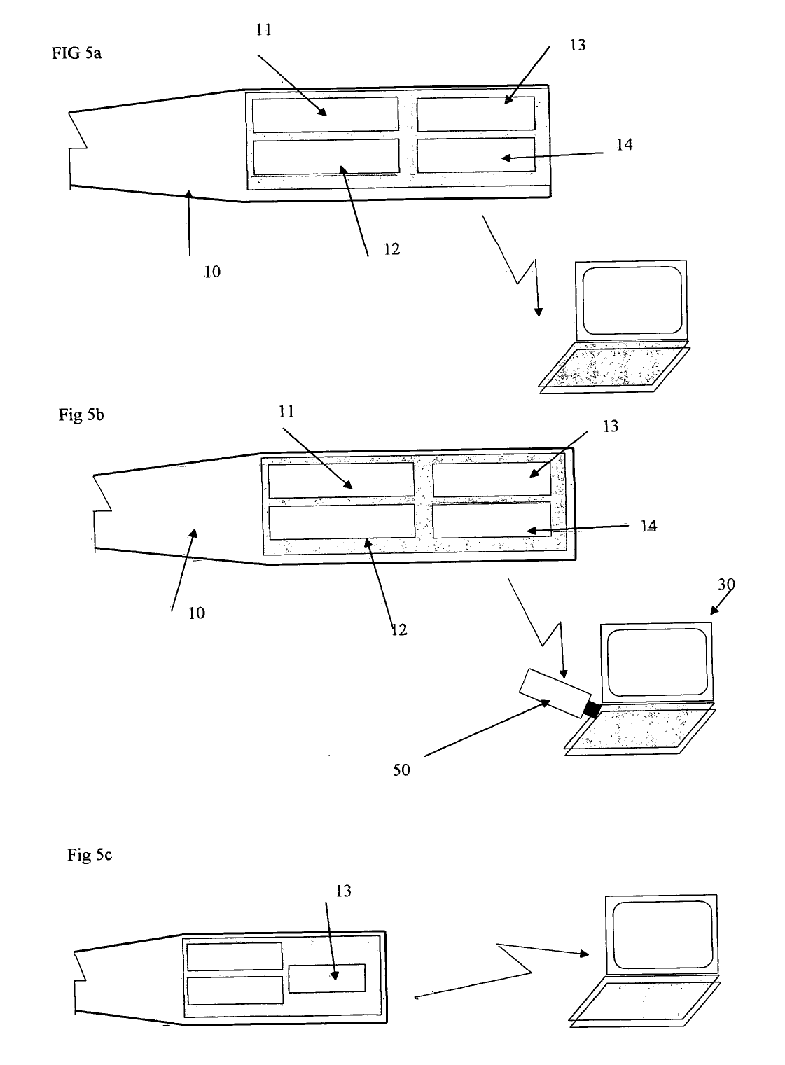

FIG. 1 represents placed in a handle of a fork a hand feeding move measure device (10) including a move sensor (11), a processing data device (12) which is a chip and a device of transmission device (13) to a distant terminal (30), FIG. 3. This hand feeding move processing device (10) unit is a device which measures, processes and transmits relevant mealtime hand moves data of food (F) brought into the mouth (M) of the user. This device can also take place in a watch or a peripheral specifically designed for this purpose. FIG. 2A represents a chewing measure device (20) which includes a chewing sensor (21) which can for example be a microphone, and a data processing device (22) which analyzes and filters data and then transmits the relevant data through a transmission device (23) to a terminal (30), FIG. 3. FIG. 2B represents chewing measure device (20) carried by a consumer according where the unit is placed in an auricle positioned in the ear (E) which is near the chewing zone (T) of the teeth. With this device, it is possible to measure as well as the swallowing into the digestve system (D) of chewed food (cF). The microphone being thus positioned towards the interior of the body to record and measure the noises inside the mouth, while a traditional auricle directs it towards the outside to record the noises and sounds of the surrounding external life. Being well understood that the chewing and swallowing device can be positioned elsewhere such as for example in a pendentive or in a collar resting on the coasts of the chest, the sounds or the vibrations in this case being in particular transmitted by the osseous system. FIG. 3 represents a feeding move data circuit according to the invention, including a hand feeding move measure device (10) and a chewing and swallowing measure device (20) which transmit the feeding measured datas to the terminal (30). The terminal (30) is a computer or a mobile phone or a personal assistant specifically designed for this purpose. The terminal is provided with a treatment chip and its operating software. In a case of FIG. 3, the terminal (30) being in network with a Internet gate (40) on which other users (80) also come to carry out the analysis of their feeding kinetics of their various meals. All the users which can take part together in a course or a forum. The users can there be advised and followed up by a professor or by their comrade of class to modify their feeding body moves in order to progress in good mealtime practices. FIG. 4 represents a data restitution principle which includes a first type of curve, the hand move analysis (41a, 41b) curve which represents for example the gestures of brought food to the mouth with the fork, a second type of curve, the chewing analysis (42i) curve, and a third type of curve, the swallowing analysis (43) curve. The movement of arm (100) or chewing and swallowing (200) being represented in a separate way but being correlated one with the other so that the user can correct one or the other of its parameters. A characteristic of the invention being by the plurality of the catches of measurements to offer a better filtering of the collected data. Indeed, it is understood that a user moves his arm for feeding his mouth as well as for gestures of body expression or of seized objects, gestures which do not correspond to a gesture of brought food in the mouth. The invention by correlating the gestures of movements of brought (100) of the arm to those of the chewing (200) thus makes it possible to filter the gestures and to retain as gesture of feeding the mouth only the gestures which are followed in a lapse of time by chewing. The terminal (30) which can be equipped with a real time alarm which makes the user informed during the meal if the oves are in the acceptable curve of movement (100, 200) according to the progression program. In this case the alarm can be visual or through sound and informs the user he must modify his moves, either by slowing down (41i) its gestures or by increasing their repetition (42i). Alarm being able to be positioned on the terminal (30) or on the move measure devices (11, 21) or on another distant periphical specific object on the table at the time of the meal. The FIGS. 5a, 5b and 5c represent three more particular forms of connection between the move measure devices (10, 20) and the terminal (30). The FIGS. 5a, 5b and 5c representing in this case a fork, but the case of figure applying just as easily to an auricle or pendentive. The FIG. 5a represents a fork handle feeding move measure device (10) including a chart on which is mounted a sensor (11), a micro controller (12) and one device of transmission (13) with its antenna (14) connected by wave according to a known protocol of type WIFI, Bluetooth or by simple radio frequency with the terminal (30) which in this case is a portable computer. The FIG. 5b represents the same handle of fork (10) as that of the FIG. 5a, with in this case a connection by wave as according to the FIG. 5a, but with in this case a terminal (30) which is not even equipped with the software of treatment, this one being placed in a key (50) for example of type USB, known type, which is connected on any computer. This solution giving even more mobility and of lightness to the user. The FIG. 5c represents a third type of solution in which the mode of transmission is infra red (13) of known type.

The invention relates to a meal time eating regulation device characterized in that it includes a hand move measure device (10) of food to the mouth, including a move sensor (11), the data collected being treated and being filtered by a processing data device (12) and transmitted by a transmission device (13) to a terminal (30) wich gives a teaching feedback restitution (100, 200) of these data to the user so as to inflect the moves and/or the kinetics of his body in the meal time.

The invention relates to a meal time eating regulation device characterized in that it includes a chewing and/or swallowing measure device (20) which is in connection with the processing data device (12)

The invention relates to a meal time eating regulation device characterized in that the hand move measure device (10) of food to the mouth and the chewing and/or swallowing measure device (20) datas are cross analysed by a processing data device (12)

The invention relates to a meal time eating regulation device characterized in that it includes a real time warning device skeletal which follows the progression of the user.

What I claim as my invention is:

1. Meal time eating regulation device characterized in that it includes a hand move measure device (10) of food to the mouth, including a move sensor (11), the data collected being treated and being filtered by a processing data device (12) and transmitted by a transmission device (13) to a terminal (30) which gives a teaching feedback restitution (100, 200) of these data to the user so as to inflect the moves and/or the kinetics of his body in the meal time.

2. Meal time eating regulation device according to claim 1 characterized in that it which includes a chewing and/or swallowing measure device (20) which is in connection with the processing data device (12)

3. Device of regulation of the food according to claim 1 and to claim 2 characterized in that the hand move measure device (10) of food to the mouth and the chewing and/or swallowing measure device (20) datas are cross analysed by a processing data device (12)

4. Meal time eating regulation device according to claim 1 characterized in that it includes a real time warning device skeletal which follows the progression of the user.
